(19) United States
(12) Patent Application Publication
(10) Pub. No.: US 2016/0208299 A1
(43) Pub. Date: Jul. 21, 2016

(54) PRODUCTION OF ISOQUINOLINE ALKALOIDS AND FLAVONOIDS IN PLANT CELL CULTURES OF GOLDENSEAL (HYDRASTIS CANADENSIS)

(71) Applicant: DIANAPLANTSCIENCES INC., Portland, OR (US)

(72) Inventors: Raymond E.B. Ketchum, Tidard, OR (US); Colby G. Caldwell, Oregon City, OR (US); Sung-Yong H. Yoon, Lake Oswego, OR (US)

(21) Appl. No.: 14/996,411

(22) Filed: Jan. 15, 2016

Related U.S. Application Data
(60) Provisional application No. 62/104,259, filed on Jan. 16, 2015.

(51) Int. Cl. C12P 17/12; A61K 36/71

(52) U.S. Cl. CPC C12P 17/12; A61K 36/71

(57) ABSTRACT

Natural product preparations of isoquinoline alkaloids are obtained from plant callus cells of goldenseal, Hydrastis canadensis, grown in suspension cell culture. The natural product preparations can be extracted from the cells and/or obtained from the culture medium in which the cells are grown. Accordingly, callus cell lines can be selected to grow in suspension culture, produce desirable levels and/or ratios of isoquinoline alkaloids, and/or release the isoquinoline alkaloids into the liquid medium. The level and/or ratio of certain isoquinoline alkaloids, such as hydrastine and/or berberine produced by the cells and/or released into the medium can be different than those found in native plants or plant parts (e.g., rhizomes) from which the cells were derived. For instance, cell lines can be selected to increase the production and/or ratio of hydrastine to berberine, or vice versa.

### CROSS-REFERENCE TO RELATED APPLICATIONS

The present application claims the benefit of priority to U.S. Provisional Application No. 62/104,259, filed Jan. 16, 2015, entitled “PRODUCTION OF ISOQUINOLINE ALKALOIDS AND FLAVONOIDS IN PLANT CELL CULTURES OF GOLDENSEAL (HYDRASTIS CANADENSIS),” the entire contents of each of which is incorporated herein by specific reference.

### BACKGROUND

1. Technical Field

This disclosure relates to natural product preparations and methods for producing the same. Specifically, embodiments of the present disclosure include an isoquinoline alkaloid natural product preparation, (callus) cell lines and cell cultures selected to produce the same, and methods for producing and/or recovering the same from cells, medium, or both, of a suspension cell culture.

2. Related Technology

Hydrastis canadensis, commonly known as goldenseal, but less commonly and historically known as yellow root, orange root, yellow puccoon, ground raspberry, wild curcuma, turmeric root, Indian dye, eye root, eye balm, Indian paint, jaundice root, and Warnera, has a history as a natural plant remedy for a variety of ailments. The rhizome, in particular, was used by Native Americans as a tonic, treatment for stomach and liver disorders, eye infections, mouth sores, ulcers, hemostatic, and as a broad spectrum treatment against bacterial and protozoan infections. It began to become scarce in North America as early as 1850 and by 1905 the US Department of Agriculture issued a bulletin (No. 51) addressing the increasing demand of goldenseal. Goldenseal has since been protected from export under Appendix II of CITES. It is listed as threatened or endangered in 12 of the states in which it is considered native. Accordingly, much of the research on plant cell culture of goldenseal has focused on regeneration and micropropagation for increasing plant numbers.

Goldenseal is a member of the buttercup family, Ranunculaceae. Its basal leaves emerge from the rhizome on long petioles. Each serrated leaf is broad and palmately 5-9 lobed, with two leaves at the end of each stem. The flower emerges from the summit of the stem, subtended by one of the leaves. The greenish-white flowers have no petals, but numerous filaments. The carpels eventually ripen into a head of red berries that resembles a raspberry.

Goldenseal produces many (isoquinoline) alkaloids. One of the most abundant of these alkaloids is berberine, an isoquinoline. Much of the efficacy of goldenseal has been attributed to the isoquinoline alkaloids produced by the plant, especially berberine. Berberine has been demonstrated to be effective against several different types of parasites, including Entamoeba histolytica, Giardia lamblia, and Trichomonas vaginalis. Researchers have found that berberine was effective in treating infection by blocking the adhesion of E. coli to cells lining the walls of the urinary tract. The alkaloid fraction of goldenseal, up to 6% of the dry weight of the rhizome, is thought to be the main active fraction against pathogens. However, several studies have shown that whole plant extracts are more efficacious than the isolated alkaloids. This has led to the hypothesis that goldenseal contains other compounds that act synergistically with the alkaloids.

Berberine and isoquinoline are alkaloids produced in many different plants and whose biosynthesis has been studied (FIG. 1). Less well understood is the biosynthesis of hydrastine which, without being bound to any theory, occurs exclusively in goldenseal in a defined ratio with berberine. Studies have demonstrated that hydrastine and berberine may even originate from a common precursor in goldenseal, but it is unknown whether berberine and hydrastine arise by independent or similar pathways. Regardless of their biosynthetic pathways, hydrastine and canadine and derivative alkaloids are uniquely produced by goldenseal and are diagnostic for authentication of goldenseal root in commercial preparations. Thus, while a variety of plants may produce berberine, goldenseal is the only known natural plant source of hydrastine and canadine.

### BRIEF DESCRIPTION OF THE DRAWINGS

FIG. 1 illustrated biosynthesis of berberine from (S)-reticuline. BBE—berberine bridge enzyme; SOMT—scoulerine 9-O-methyl transferase; CAS—canadine synthase; STOX—(S)-tetrahydroprotoberberine oxidase;

FIG. 2 shows structures of isoquinoline and several isoquinoline alkaloids produced by goldenseal plants and/or cell cultures;

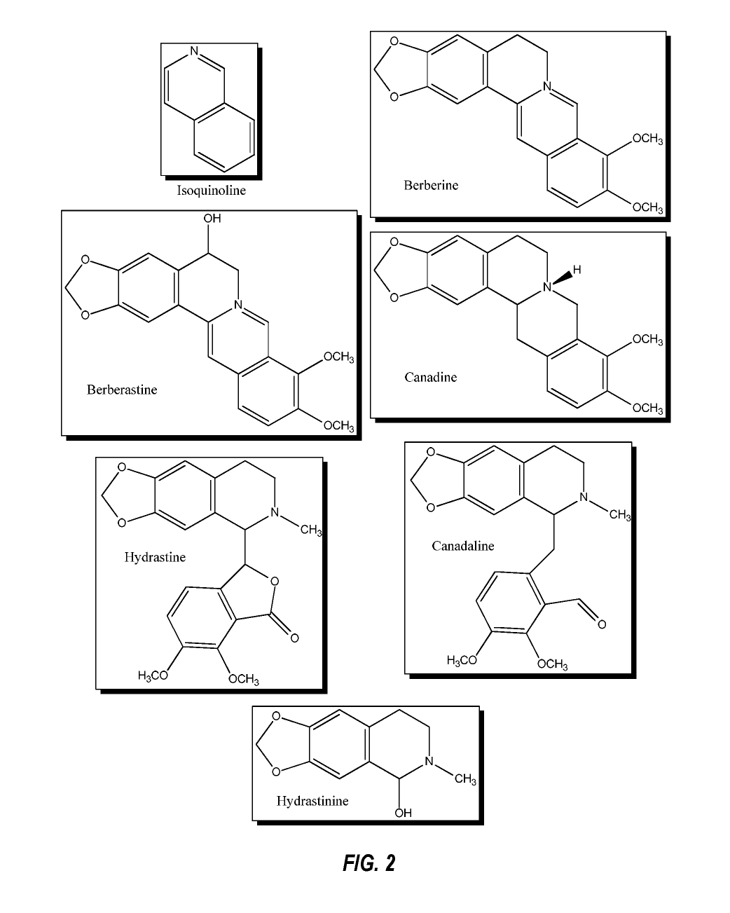

FIG. 3 illustrates extracted ion chromatograms (HPLC-MS method) for berberine (336 m/z) and hydrastine (384 m/z), in callus suspension cells derived from goldenseal and sampled shortly after initiation of cultures and before selection had begun (A), and extract of commercially available dried goldenseal rhizome (B);

FIG. 4 shows UV (diamonds) and extracted ion mass (squares) calibration curves for hydrastine (A) and berberine (B) over typical concentration range for extracts whether analysis is by HPLC or plate spectrophotometer;

FIG. 5 shows extracted ion mass chromatogram for berberine (336 m/z) and hydrastine (384 m/z) in suspension cell culture medium of callus derived from goldenseal and sampled after selection according to an exemplary embodiment of the present disclosure;

FIG. 6 shows extracted ion mass chromatogram for berberine (336 m/z) and hydrastine (384 m/z) in suspension cell culture medium of callus derived from goldenseal and sampled after selection according to another exemplary embodiment of the present disclosure;

FIG. 7 shows extracted ion mass chromatogram for berberine (336 m/z) and hydrastine (384 m/z) in suspension cell culture medium of callus derived from goldenseal and sampled after selection according to still another exemplary embodiment of the present disclosure;

FIG. 8 shows extracted ion mass chromatogram for berberine (336 m/z) and hydrastine (384 m/z) in suspension cell culture medium of callus derived from goldenseal and sampled after selection according to still another exemplary embodiment of the present disclosure;

FIG. 9 shows extracted ion mass chromatogram for berberine (336 m/z) and hydrastine (384 m/z) in suspension cell culture medium of callus derived from goldenseal and sampled after selection according to still another exemplary embodiment of the present disclosure; and

FIG. 10 shows a set of extracted ion mass chromatograms for berberine (336 m/z) and hydrastine (384 m/z) in suspension cell culture medium of callus derived from goldenseal and sampled after selection according to still another exemplary embodiment of the present disclosure (A), and in commercial goldenseal rhizomes (B).

### DETAILED DESCRIPTION

Before describing the present disclosure in detail, it is to be understood that this disclosure is not limited to the description of the particularly exemplified systems, methods, and/or products that may vary from one embodiment to the next. Thus, while certain embodiments of the present disclosure will be described in detail, with reference to specific configurations, parameters, features (e.g., components, members, elements, parts, and/or portions), etc., the descriptions are illustrative and are not to be construed as limiting the scope of the claimed invention. In addition, the terminology used herein is for the purpose of describing the embodiments, and is not necessarily intended to limit the scope of the claimed invention.

Unless defined otherwise, all technical and scientific terms used herein have the same meaning as commonly understood by one of ordinary skill in the art to which the present disclosure pertains.

Various aspects of the present disclosure, including systems, processes, and/or products may be illustrated with reference to one or more embodiments or implementations, which are exemplary in nature. As used herein, the terms “embodiment” and “implementation” mean “serving as an example, instance, or illustration,” and should not necessarily be construed as preferred or advantageous over other aspects disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used herein, the term “systems” also contemplates devices, apparatus, compositions, assemblies, kits, and so forth. Similarly, the term “process” also contemplates methods, procedures, steps, and so forth. Moreover, the term “products” also contemplates devices, apparatus, compositions, assemblies, kits, and so forth.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

It will be noted that, as used in this specification and the appended claims, the singular forms “a,” “an” and “the” include plural referents unless the context clearly dictates otherwise. Thus, for example, reference to a “callus culture” includes one, two, or more callus cultures. Similarly, reference to a plurality of referents should be interpreted as comprising a single referent and/or a plurality of referents unless the content and/or context clearly dictate otherwise. Thus, reference to “callus cultures” does not necessarily require a plurality of such callus cultures. Instead, it will be appreciated that independent of conjugation; one or more callus cultures are contemplated herein.

To facilitate understanding, like references (i.e., like naming of components and/or elements) have been used, where possible, to designate like elements common to the figures. Specifically, in the exemplary embodiments illustrated in the figures, like structures, or structures with like functions, will be provided with similar reference designations, where possible. Specific language will be used herein to describe the exemplary embodiments. Nevertheless it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

It will also be appreciated that where a range of values (e.g., less than, greater than, at least, and/or up to a certain value, and/or between two recited values) is disclosed or recited, any specific value or range of values falling within the disclosed range of values is likewise disclosed and contemplated herein. Thus, disclosure of an illustrative measurement or amount less than or equal to about 10 units or between 0 and 10 units includes, illustratively, a specific disclosure of: (i) a measurement or amount of 9 units, 5 units, 1 units, or any other value between 0 and 10 units, including 0 units and/or 10 units; and/or (ii) a measurement or amount between 9 units and 1 units, between 8 units and 2 units, between 6 units and 4 units, and/or any other range of values between 0 and 10 units.

The headings used herein are for organizational purposes only and are not meant to be used to limit the scope of the description or the claims.

Plant cell culture technology provides an alternative source for the production of desirable plant natural products. Without being bound to any theory, growing plant cells in defined nutrient media in a controlled environment provides for year-round production and batch-to-batch consistency of product. Plant cells grown in the laboratory are not exposed and/or susceptible to disease or changes in the weather and have an absolute requirement for growth under sterile conditions. Thus, the end-product is also free of disease or contamination by microorganisms. Plant cell culture allows the unlimited production of biomass from a limited amount of starting material, theoretically from a single cell. Thus, plant cells can be grown of a rare or endangered plant without sacrificing a single individual of that species or without disturbing a critical habitat. Further, plant cell culture technology enables the selection of cell lines that have an enhanced ability to produce desired compounds while undesirable compounds, such as those that impart objectionable odor or taste, are not produced in significant amounts.

Disclosed herein are natural product preparations of isoquinoline alkaloids from cells of goldenseal, Hydrastis canadensis, that are configured to grow in a callus culture on a solid medium or as a suspension culture in a liquid medium, and methods for obtaining and/or recovering the same (e.g., from the cells, medium, or both). Embodiments include (isolated and/or (undifferentiated) callus) cells or cell lines (of goldenseal, Hydrastis canadensis), including those selected to produce specific secondary metabolites (e.g., alkaloids, such as hydrastine, berberine, etc.) in amounts, at concentrations, or at ratios not found in live plants or unselected (control) cells. The cells can also be selected for growth and production of secondary metabolite(s) in suspension cell culture and, optionally, selected for extracellular release of the secondary metabolite(s) into the liquid culture medium (e.g., for extraction, isolation, and/or purification). Embodiments can also include suspension cell cultures that include the selected cells, extracts of the cells, cell culture, and/or liquid medium, as well as methods for extracting the same.

The cells can be derived from one or more plant parts, such as the inflorescence (petal, anther, stamen, pollen, etc.), seed, embryo, cotyledon, hypocotyls, fruit, vegetative tissue (any portion of the stem, leaf or cells or tissues associated with vegetative tissues), or any other portion of the plant attached to these tissues, including but not limited to roots, rhizomes, modified stems, or underground storage tissues (tubers, etc.)

The cells can be adapted to grow to a high density for a first period of time (e.g., about 3 to 28 days). For instance, the cells can be adapted for relatively fast growth and/or low production of one or more primary or secondary metabolites in a first growth medium. In a second stage, the cells can be adapted to produce high concentrations of primary or secondary metabolites, such as isoquinoline alkaloids. For instance, the cells can be adapted for relatively slow growth and/or high production of the metabolite(s) in a second production medium.

One or more embodiments of the present disclosure include one or more plant cells, cell cultures, callus and/or cell lines that are or have been selected to grow in suspension cell culture and to produce and release one or more isoquinoline alkaloids into the liquid suspension cell culture medium. In some embodiments, at least a portion of the liquid culture medium can be harvested and, optionally, replaced with (additional and/or fresh) medium. Released isoquinoline alkaloids can be extracted, isolated, and/or purified from harvested culture medium.

Over an extended period of cell culture, for example, the cell or cell culture can produce substantial amounts of one or more isoquinoline alkaloids. Accordingly, the selected callus cells may not contain (e.g., produce and retain) extraordinary or even increased amounts (or ratios) of the one or more isoquinoline alkaloids found therein (e.g., to be extracted from the cells), relative to the natural plant or unselected callus cells from which the selected callus cells were derived. Instead, the callus cells can be selected for perfusion-style suspension cell culture in which the isoquinoline alkaloids are produced in the cells at natural plant (or even decreased) levels, amounts, rates, and/or ratios, but released into the medium. Accordingly, the selected cells can produce, over an extended period of time, substantially more of the isoquinoline alkaloids that are produced in the natural plant or unselected callus cells from which the selected callus cells were derived.

Certain embodiments of the present disclosure, however, include one or more plant cells, cell cultures, callus and/or cell lines that are or have been selected to produce one or more isoquinoline alkaloids at an amount, concentration, or ratio other than that found in the natural plant from which it was derived. The amount and/or ratio of berberine and/or hydrastine can be measured and/or determined by cellular and/or (liquid) media content. For instance, one or more callus cell lines can be selected to produce one or more isoquinoline alkaloids in (liquid suspension) cell culture at an amount, concentration, or ratio higher or lower than that found in the natural plant from which it was derived. In at least one embodiment, producing isoquinoline alkaloids can include forming the isoquinoline alkaloids, retaining the isoquinoline alkaloids within the cells, and/or releasing the isoquinoline alkaloids into the (liquid suspension) cell culture medium. Solid and other media are also contemplated herein.

In certain embodiments, the suspension cell culture and/or callus cells can (be selected to) produce (and optionally release into the medium) one or more isoquinoline alkaloids (e.g., berberine and/or hydrastine) in suspension cell culture in amounts greater than, less than, or equal to those found in the natural plant from which the callus cells were derived. For instance, a cell line of callus cells can be selected to have or exhibit increased production of one or more isoquinoline alkaloids compared to the tissue of the natural plant from which it was derived. In some embodiments, the selected cells or cell line can be selected to have or exhibit about a 5%, 8%, 10%, 12%, 15%, 18%, 20%, 25%, 30%, 40%, 50%, 60%, 70%, 80%, 90%, 95%, 100%, 125%, 140%, 150%, 175%, 200%, 250%, 280%, or more increase in the production of one or more isoquinoline alkaloids (e.g., berberine and/or hydrastine) compared to the tissue of the natural plant or unselected callus (cells) from which it was derived, or within a range of the foregoing values, on a dry weight basis.

In at least one embodiment, the suspension cell culture and/or callus cells thereof can (be selected to) produce (and optionally release into the medium) hydrastine at up to, at least, greater than, less than, between, or about 1 mg/L, 2 mg/L, 3 mg/L, 5 mg/L, 8 mg/L, 10 mg/L, 15 mg/L, 20 mg/L, 25 mg/L, 30 mg/L, 35 mg/L, 40 mg/L, 45 mg/L, 50 mg/L, 55 mg/L, 60 mg/L, 80 mg/L, 100 mg/L, 150 mg/L, 180 mg/L, 200 mg/L, 250 mg/L, 275 mg/L, 282 mg/L, 300 mg/L, 325 mg/L, 326 mg/L, 350 mg/L, 400 mg/L, 450 mg/L, 500 mg/L, or more, of suspension cell culture, packed cells, and/or culture medium, or within a range of the foregoing values. A dry (e.g., freeze-dried) mass of the selected cells (e.g., grown in suspension cell culture) can comprise up to, at least, greater than, less than, between, or about 10 mg/g (10 ug/mg or 1% DW), 15 mg/g, 20 mg/g, 25 mg/g, 30 mg/g, 35 mg/g, 40 mg/g, 50 mg/g, 55 mg/g, 56 ug/mg, 60 mg/g, 65 mg/g, 70 mg/g, 75 mg/g, 80 mg/g, 85 mg/g, 90 mg/g, 95 mg/g, 100 mg/g, or more hydrastine DW. Accordingly, the (dry mass of the) cells can comprise up to, at least, greater than, less than, between, or about 1%, 1.5%, 2%, 2.5%, 3%, 3.5%, 4%, 4.5%, 5%, 5.5%, 5.6%, 6%, 6.5%, 7%, 7.5%, 8%, 8.5%, 9%, 9.5%, 10%, or more hydrastine (on a dry weight basis; DW, w/w, or w/v).

In some embodiments, the suspension cell culture and/or callus cells thereof can (be selected to) produce (and optionally release into the medium) berberine at up to, at least, greater than, less than, between, or about 1 mg/L, 2 mg/L, 3 mg/L, 5 mg/L, 8 mg/L, 10 mg/L, 15 mg/L, 20 mg/L, 25 mg/L, 30 mg/L, 35 mg/L, 40 mg/L, 45 mg/L, 50 mg/L, 55 mg/L, 60 mg/L, 100 mg/L, 150 mg/L, 200 mg/L, 250 mg/L, 282 mg/L, 300 mg/L, 500 mg/L, 1 g/L, 2 g/L, 2.3 g/L, 2.5 g/L, 5 g/L, 10 g/L, or more, of suspension cell culture, packed cells, and/or culture medium, or within a range of the foregoing values. A dry (e.g., freeze-dried) mass of the selected cells (e.g., grown in suspension cell culture) can comprise up to, at least, greater than, less than, between, or about 10 mg/g (10 ug/mg or 1% DW), 15 mg/g, 20 mg/g, 25 mg/g, 30 mg/g, 35 mg/g, 40 mg/g, 50 mg/g, 55 mg/g, 60 mg/g, 65 mg/g, 70 mg/g, 75 mg/g, 80 mg/g, 85 mg/g, 90 mg/g, 95 mg/g, 100 mg/g, or more berberine DW. Accordingly, the (dry mass of the) cells can comprise up to, at least, greater than, less than, between, or about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, or more berberine (on a dry weight basis; DW, w/w, or w/v).

Without being bound to any theory, goldenseal plant (rhizomes) typically contains 2.5% to 6% total alkaloids (on a dry weight basis). Of the total alkaloids, up to or about 2% of the dry mass can be comprised of hydrastine and up to or about 2.5% of the dry mass can be comprised of berberine (or about 4.5% total hydrastine plus berberine). In one or more embodiments, the suspension cell culture and/or callus cells thereof can (be selected to) produce (and optionally release into the medium) greater than about 2% hydrastine and/or 2.5% berberine, or about 4.5% total hydrastine and berberine, DW. Accordingly, the combination of hydrastine and berberine in the suspension cell culture, liquid medium, and/or callus cells thereof can be (selected to be) greater than about 1%, 2%, 3%, 4%, 4.5%, 5%, 5.5%, 5.6%, 6%, 6.5%, 7%, 7.5%, 8%, 8.5%, 9%, 9.5%, 10%, 10.5%, 11%, 11.5%, 12%, or more total hydrastine plus berberine (on a dry weight basis; DW, w/w, or w/v). In addition, while the cells can contain a specific dry weight amount or percentage of specific alkaloids (e.g., hydrastine and/or berberine), the liquid culture medium can contain substantially more of the specific alkaloids.

Without being bound to any theory, goldenseal plants and/or rhizomes, roots, or other portion thereof are known to produce hydrastine and berberine at a ratio of approximately 2:2.5 (hydrastine:berberine), on a dry weight basis. Certain embodiments of the present disclosure, however, can comprise callus derived from a natural goldenseal plant and selected to produce hydrastine and berberine (e.g., in suspension cell culture) at a ratio other than that found in or produced by the natural plant (from which the callus was derived). For instance, the hydrastine:berberine ratio produced by the callus (e.g., in suspension cell culture) can be greater than or less than the hydrastine:berberine ratio found in or produced by the natural plant from which the callus was derived. In at least one embodiment, for example, the hydrastine:berberine ratio produced by the callus (e.g., in suspension cell culture) can be up to, at least, less than, greater than, or equal to about 1:1, 2:1.5, 1.4:1, 2:1, 2.5:1, 3:1, 4:1, 5:1, 6:1, 7:1, 8:1, 9:1, 10:1, 11:1, 12:1, 13:1, 14:1, 15:1, 16:1, 18:1, 20:1, or more, or within a range of the foregoing values. Alternatively, the hydrastine:berberine ratio produced by the callus (e.g., in suspension cell culture) can be up to, at least, less than, greater than, or equal to about 1:1, 1:1.5, 1:2, 1:2.5, 1:3 (or 0.33), 1:4, 1:5, 1:6 (or 0.16), 1:7, 1:8, 1:9, 1:10 (or 0.1), 1:20, 1:30, 1:40, 1:50, or 1:60 (or 0.016), 1:80, 1:100, or less, or within a range of the foregoing values in some embodiments.

In some embodiments, the ratio of hydrastine:berberine produced by the cells (in suspension culture), retained therein, and/or released into the cell culture medium can be greater than or equal to about 0.9, preferably greater than or equal to about 0.95, more preferably greater than or equal to about 1.0 (1:1), still more preferably greater than or equal to about 1.5, still more preferably greater than or equal to about 2.0 (2:1), still more preferably greater than or equal to about 2.5, still more preferably greater than or equal to about 3.0, still more preferably greater than or equal to about 3.5, still more preferably greater than or equal to about 4.0, still more preferably greater than or equal to about 4.5, still more preferably greater than or equal to about 5.0, still more preferably greater than or equal to about 5.5, still more preferably greater than or equal to about 6.0, still more preferably greater than or equal to about 6.5, still more preferably greater than or equal to about 7.0, still more preferably greater than or equal to about 8.0, still more preferably greater than or equal to about 9.0, still more preferably greater than or equal to about 10, still more preferably greater than or equal to about 11, still more preferably greater than or equal to about 12, still more preferably greater than or equal to about 13, still more preferably greater than or equal to about 14, still more preferably greater than or equal to about 15, still more preferably greater than or equal to about 16, or more, or within a range of the foregoing values.

In other embodiments, the ratio of hydrastine:berberine produced by the cells (in suspension culture), retained therein, and/or released into the cell culture medium can be less than or equal to about 0.75, preferably less than or equal to about 0.5, more preferably less than or equal to about 0.33 (1:3), still more preferably less than or equal to about 0.25 (1:4), still more preferably less than or equal to about 0.2, still more preferably less than or equal to about 0.167, still more preferably less than or equal to about 0.14, still more preferably less than or equal to about 0.125, still more preferably less than or equal to about 0.11, still more preferably less than or equal to about 0.1, still more preferably less than or equal to about 0.075, still more preferably less than or equal to about 0.05, still more preferably less than or equal to about 0.033, still more preferably less than or equal to about 0.025, still more preferably less than or equal to about 0.02, still more preferably less than or equal to about 0.0167, or less, or within a range of the foregoing values.

Furthermore, in some embodiments, the callus cells can (be selected to) produce berberine and hydrastine in suspension cell culture at sufficient levels to produce medium comprising more hydrastine than berberine, or vice versa. For instance, the callus cells can (be selected to) release berberine and hydrastine into suspension cell culture medium. Accordingly, the cells of the suspension cell culture can be selected to produce and optionally release: (i) up to, at least, greater than, equal to, or between about 1.5-times, 2-times, 3-times, 4-times, 5-times, 6-times, 7-times, 8-times, 9-times, 10-times, 15-times, 20-times, 30-times, 40-times, 50-times, and/or 60-times more berberine than hydrastine (into the medium); (ii) up to, at least, about, greater than, equal to, or between 1.5-times, 2-times, 2.5-times, 3-times, 3.5-times, 4-times, 4.5-times, 5-times, 5.5-times, 6-times, 6.5-times, 7-times, 8-times, 9-times, 10-times, 12-times, 15-times, 16-time, 18-times, or 20-times more hydrastine than berberine (into the medium); or (iii) approximately equal amounts of hydrastine and berberine (into the medium), illustratively, or within a range of the foregoing values.

Without being bound to any theory, it is noted that in certain embodiments, the amount of alkaloids (or ratio thereof) or other secondary metabolite(s) in the liquid suspension cell culture medium can affect the growth and/or production profile of the cell culture and/or cells thereof. For instance, suspension cell cultures may not be selectable above a certain amount of production due to the need for viability of the growing culture. Accordingly, the amounts and/or ratios of alkaloids described herein can be or comprise amounts and/or ratios in a viable suspension cell culture. For instance, in at least some embodiments, the (selected) callus cells can be growing in suspension cell culture and producing and releasing the described the amounts and/or ratios of alkaloids into the medium while maintaining viability and/or production continuity of the suspension cell culture.

Without being bound to any theory, modern methods of analysis can employ liquid chromatography with either UV detection or mass spectral (MS) detection. The United States Pharmacopoeia (USP) (or United States Pharmacopeial Convention) methods employing thin-layer chromatography (TLC) and high performance liquid chromatography (HPLC) are known. Up to 28 different alkaloids have been reported from goldenseal rhizome using liquid chromatography-mass spectrometry (LC-MS), or HPLC-MS methods. In addition to providing accurate quantification, LC-UV-MS methods provide enough structural information to give confidence in the identities of the compounds being analyzed. In some embodiments of the present disclosure, the ratio of hydrastine:berberine produced by the cells (in suspension culture), retained therein, and/or released into the cell culture medium can be measured using LC-MS or HPLC-MS detection. The ratio of hydrastine:berberine can also be measured by LC-UV or HPLC/UV or other methods in some embodiments. Without being bound to any theory, such ratios can vary up to, more than, or about 10-fold depending on detection method in certain embodiments.

Without being bound to any theory, the USP methods specify a minimum of (not less than) 2.5% berberine and 2% hydrastine for commercial goldenseal (root) (calculated on a dry weight basis). Accordingly, to adhere with USP standards, commercially inferior and/or inadequate goldenseal, or (naturally occurring) extracts or preparations thereof, may be adulterated with purified berberine and/or hydrastine (e.g., from Coptis sp. or other plants, synthetic compounds, etc.) to reach the industry standard and/or minimum requirements.

Because of the additional expense associated with the adulteration and/or supplementation, however, those skilled in the art have not been motivated to adulterate and/or supplement goldenseal, or (naturally occurring) extracts or preparations thereof with berberine and/or hydrastine to substantially exceed the industry standard and/or minimum requirements. For instance, without being bound to any theory, goldenseal (Hydrastis canadensis) is the only known source of naturally-occurring hydrastine. Accordingly, goldenseal hydrastine, in particular, can be prohibitively expensive to obtain for supplementing natural product preparations, such as extracts. Thus, goldenseal extracts of the present disclosure (e.g., having significantly more than, for example, 2.5% berberine and/or 2% hydrastine, DW) can provide an advantage to consumers and producers of goldenseal natural products and preparations thereof.

Certain embodiments of the present disclosure include a (pure and/or unadulterated) goldenseal extract comprising purified goldenseal alkaloids. The extract can be in substantial dry (e.g., powder, crystal, etc.) form. In at least one embodiment, the extract is (substantially) free of one or more non-goldenseal alkaloids, (e.g., hydrastine and/or berberine). The extract can include greater than or equal to about 2%, 2.1%, 2.2%, 2.3%, 2.4%, 2.5%, 2.6%, 2.7%, 2.8%, 2.9%, 3%, 3.2%, 3.5%, 3.8%, 4%, 4.2%, 4.5%, 4.8%, 5%, 5.2%, 5.5%, 5.6%, or more (pure and/or unadulterated) goldenseal hydrastine on a dry weight basis. The extract can also (or alternatively) include greater than or equal to about 2.5%, 2.6%, 2.7%, 2.8%, 2.9%, 3%, 3.2%, 3.5%, 3.8%, 4%, 4.2%, 4.5%, 4.8%, 5%, 5.2%, 5.5%, 5.6%, or more (pure and/or unadulterated) goldenseal berberine on a dry weight basis. The extract can also (or alternatively) include greater than or equal to about 4.5%, 4.8%, 5%, 5.2%, 5.5%, 6%, 6.5%, 7%, 7.5%, 8%, 8.5%, 9%, 9.1% or more (pure and/or unadulterated) goldenseal hydrastine plus berberine on a dry weight basis.

In some embodiments, the ratio of hydrastine:berberine produced by the cells (in suspension culture), retained therein, released into the cell culture medium, and/or extracted therefrom can be a pure and/or unadulterated ratio in certain embodiments of the present disclosure. For instance, a cell extract, culture media extract, and/or natural product preparation can have any of the foregoing ratios without being supplemented with adulterated, heterologous, and/or synthetic hydrastine and/or berberine. Thus, the callus cells or elite cell lines of the present disclosure can be selected and/or adapted to produce goldenseal hydrastine and berberine (e.g., in suspension cell culture) at a ratio sufficient to produce a (pure) goldenseal natural product preparation having one or more of the foregoing unadulterated ratios of hydrastine:berberine. The goldenseal natural product preparation obtained therefrom can, therefore, comprise natural or naturally-occurring goldenseal hydrastine and berberine having one or more of the foregoing unadulterated ratios. In some embodiments, the callus cells or elite cell lines of the present disclosure can be selected and/or adapted to produce (and release into (liquid) culture medium) hydrastine and berberine at a ratio sufficient to produce a (pure) goldenseal natural product preparation having one or more of the foregoing unadulterated ratios of hydrastine:berberine.

An illustrative method of producing a natural product preparation can include establishing callus and/or a callus culture derived from tissue of a natural plant (e.g., a natural plant that produces one or more isoquinoline alkaloids, such as hydrastine and/or berberine). For instance, the plant from which the callus is derived can be goldenseal (Hydrastis canadensis). The tissue can be any portion of the plant containing viable cells, including, for example, the seed, embryo, cotyledon, hypocotyls, rhizome, root, stem, leaf, bract, or any portion of the inflorescence, including sepals, petals, stamen, anther, pollen, style, pistil, or another suitable portion of the plant. The one or more isoquinoline alkaloids can include berberine and/or hydrastine, and, optionally, other alkaloids (e.g., found in goldenseal plants).

In some embodiments, (callus) cultures of goldenseal are generated from excised embryos or a part or portion thereof. In certain embodiments, plating the embryos on an agar medium supplemented with 10 μM GA₃ can improve growth. In some embodiments, callus formation was on medium containing Gamborg's B5 salts, vitamins, and micronutrients supplemented with 3% sucrose, and 1 μM NAA plus 10 μM BA for leaf explants, and 0 μM NAA plus 10 μM BA for root explants.

In another embodiment of the present disclosure, tissue culture may be developed in vitro micropropagation method from callus initiated from leaf discs cultivated on MS medium supplemented with 3% sucrose. Friable callus formation can be achieved on 5.3 μM NAA with 2.2 μM TDZ. In the absence of auxin, media containing any of the cytokinins, benzyl adenine, thidiazuron, or kinetin, at a concentration of ˜4.5 μM resulted in shoot formation from 100% of the callus cultures. A medium containing MS salts, vitamins, micronutrients, supplemented with 3% sucrose and 2.5 μM TDZ with 5 μM NAA produces shoot formation from leaves. Callus from stem explants on medium containing MS salts in NAA and in combination with BA produces mostly callus with limited regeneration.

The method can also include growing a plurality of callus cells (or callus cell lines) from the established callus. Certain embodiments can include cultivating one or more plant calli or callus cell lines (e.g., the established callus or callus derived from the established callus culture) in a defined solid or liquid medium. It will be appreciated that reference to cultivating callus in a medium also includes cultivating the callus on the medium. For instance, the method can include cultivating one or more callus cells in a defined solid or liquid medium under growth conditions in a first growth stage or for a first period of time. In the first growth stage or first period of time, the callus may grow rapidly in a growth medium (e.g., doubling in cell density in about 7 days time).

Once plant cells have been established as a callus can cultivated in culture (medium), the plant cell culture can be analyzed and the cells and/or cell line(s) can be selected to establish elite cell lines. Such selected and/or elite cell lines can exhibit increased production and/or altered production ratio of one or more (isoquinoline) alkaloids (and optionally release thereof into the culture medium) as compared to wild type plant, plant parts, or established callus cells from which the selected and/or elite cell lines were derived. Accordingly, at least one embodiment can include selecting a plant callus with increased isoquinoline alkaloid production. For instance, the method can include selecting a callus cell line (from the established and/or cultivated callus or callus culture) with increased isoquinoline alkaloid production compared to the tissue of the natural plant from which it was derived.

Some embodiments can include selecting cell suspension cultures based on the profile, amount, and/or ratio of secondary metabolites in the liquid culture medium (e.g., rather than cellular content). In some embodiments, selecting (or (cell) selection) can include monitoring the generational alterations in production and/or release into the medium of one or more (isoquinoline) alkaloids (or ratio thereof) and passaging (e.g., transferring a portion of the culture into fresh medium, replacing a portion of the culture medium, etc.) cultures that exhibit desirable changes. Such selective pressure can be consistent over an extended period of time. For instance, in certain embodiments, (isoquinoline) alkaloid production can be measured (e.g., in the liquid culture medium) every 1, 2, 3, 4, 5, 6, 7, 8, 9, 10, 11, 12, 13, or 14 days. Cultures can then be screened for further growth and selection. For instance, cultures producing greater than or equal to the amount of isoquinoline alkaloid (e.g., in the liquid culture medium) produced by the previous or parent culture can be further passaged and/or subcultured. In at least one embodiment, production can be measured prior to or on the seventh day of culture.

The elite cell lines established using embodiments of the presently disclosed (screening and other) methods can be cultured to produce desired quantities of alkaloids in cell culture. For instance, in one or more embodiments, the method can include cultivating one or more callus cells in a liquid medium to obtain a suspension cell culture. The method can include, in a second growth stage or for a second period of time, cultivating the callus from the first stage in a liquid medium to obtain a suspension cell culture. In at least one embodiment, the culture medium (e.g., a production medium) in the second stage increases production of isoquinoline alkaloids (e.g., in the callus cells and/or in the (liquid) medium) as compared to the first stage (or growth medium). Thus, the production stage can be optimized to produce certain alkaloids including, but not limited to berberine, hydrastine, dihydroberberine, and canadine. The production stage can also be optimized to produce desirable ratios of certain alkaloids, such as the ratio of hydrastine to berberine (hydrastine:berberine). Thus, the cell culture medium can be optimized for maximum alkaloid production and/or to maximize the ratio of certain alkaloids. In the second growth stage, the (selected) plant callus or callus culture may grow slowly compared to the first period of time in the growth medium and/or may produce high concentrations of isoquinoline alkaloids, which can be retained in the cells and/or released into a production medium.

Some embodiments include a method of producing a goldenseal natural product preparation (or recovering natural product from goldenseal suspension cell culture). The natural product and/or natural product preparation can have a high concentration of one or more isoquinoline alkaloids. Specifically, the natural product and/or natural product preparation can have increased isoquinoline concentration (e.g., compared to an extract or natural product of the plant from which the callus was derived). For instance, the natural product and/or natural product preparation can comprise up to about 2.3 grams berberine and/or 326 milligrams hydrastine per liter of culture medium in certain embodiments. In at least one embodiment, the natural product and/or natural product preparation can also (or alternatively) comprise callus cells having up to or greater than about 9% (e.g., 9.1%) total (pure and/or unadulterated goldenseal) hydrastine and (or plus) berberine, up to or greater than about 5.5% (e.g., 5.6%) (pure and/or unadulterated goldenseal) hydrastine, and/or up to or greater than about 3.5% (pure and/or unadulterated goldenseal) berberine, on a dry weight basis.

In certain embodiments, the natural product and/or natural product preparation can comprise hydrastine and berberine at a ratio other than that found in the tissue of the natural plant from which the callus was derived and/or than found in an extract or natural product (preparation) thereof. For instance, the natural product and/or natural product preparation can also (or alternatively) comprise (pure and/or unadulterated goldenseal) hydrastine and berberine at a ratio of greater than (or equal to) about 2:2.5, 1:1, 1.25:1, 1.4:1, 1.5:1, 2:1, or more, hydrastine:berberine, w/w. In some embodiments, the natural product and/or natural product preparation can comprise hydrastine and berberine at a ratio of up to about 16:1 (hydrastine:berberine) or down to about 1:60 (hydrastine:berberine). As indicated above, the foregoing values and ranges of values are intended to include any and every specific value and range of values falling within the disclosed ranges.

The method can also include recovering natural product from the suspension cell culture. For instance, the method can include recovering and/or harvesting the cultured cells, comprising a natural product. The method can also include recovering natural product from the cells, medium, or both, of the suspension cell culture. For instance, recovering natural product from the cells can include extracting and/or purifying the natural product (e.g., from one or more cellular components). Certain embodiments can include harvesting and/or replacing the liquid culture medium comprising the natural product. For instance, the suspension cells can release the natural product into the liquid culture medium. Accordingly, the natural product producing suspension cell culture can be maintained and the natural product harvested therefrom through recovering and replacing the liquid culture medium (e.g., with fresh (production) medium). Certain embodiments can also include a perfusion-type cell culture system.

Without being bound to any theory, in at least one embodiment, aspects of the present disclosure are predicated on the finding that callus and suspension cultures derived from plant tissue of goldenseal will, under the appropriate conditions, produce in culture one or more of the alkaloids found in the intact and/or natural plant. In a commercial goldenseal (rhizome) preparation, the concentration of hydrastine may occur in the plant in a ratio of approximately 2:2.5 with berberine on a dry weight basis (e.g., 2% hydrastine DW (or 20 milligrams of hydrastine per gram of dried goldenseal rhizomes), 2.5% berberine DW (or 25 milligrams of berberine per gram of dried goldenseal rhizomes), and, therefore, 4.5% total hydrastine plus berberine DW (or 45 milligrams of hydrastine plus berberine per gram of dried goldenseal rhizomes)). Extracts thereof typically contain the same or similar ratio of hydrastine to berberine on a dry weight basis.

The forgoing embodiments and other subject matter of the disclosure will now be described and explained with additional specificity and detail through the use of the following Examples. The following details and examples represent exemplary implementations and are not meant to limit the disclosure to the specific attributes disclosed herein.

### Examples

### Example 1

### Surface Sterilization of Plant Tissue

Hydrastis canadensis seeds and rhizomes were obtained from commercial suppliers. In some cases, seeds and rhizomes were used to obtain plants. Regardless of the source of the plant tissue, sterilization was accomplished using similar methods. Seeds or plant tissues were washed in soapy water and rinsed in tap water. Tissue was rinsed in 60% to 80% ethanol for 10 to 30 s, surface sterilized for 10 to 20 min in 1 to 3% sodium hypochlorite solution and then rinsed 3 to 5 times with sterile distilled water. Seeds were soaked in sterile deionized water overnight, and sterilized again according to the protocol given above.

The embryos were excised from the sterilized seeds under aseptic conditions and placed on callus induction medium. Sterilized explants were cut and in some cases the epidermis was removed prior to plating on a callus induction medium.

### Example 2

### Callus Induction of Hydrastis Canadensis

Sterilized tissue was placed on a basal medium such as MS, LS, B5, White's, N6, NN, Anderson's, Blaydes', Y3, QL, KM or similar medium formulation suitable for growth of plant cells; containing 1 to 6% total concentration of sucrose, glucose, fructose, or a combination of those carbohydrates; different combinations and concentrations of auxins: 2,4-D (0-10 mg L⁻¹), NAA (0-10 mg L⁻¹), IAA (0-10 mg L⁻¹), IBA (0-10 mg L⁻¹), picloram (0-10 mg L⁻¹), and cytokinins: kinetin (0-10 mg L⁻¹), zeatin (0-10 mg L⁻¹), BA (0-10 mg L⁻¹), 2ip (0-10 mg L⁻¹), and TDZ (0-10 mg L⁻¹). All media were solidified by 0.3 to 1.5% agar or purified gellan gum after adjusting the pH to 5.5 to 6 and autoclaved at 1.2 kg cm⁻² with time adjusted depending on the volume of the medium according to Burger [6]. Cultures were incubated at 18 to 30±1° C. in dark, 16/8 hrs or 8/16 hrs of light/dark, or continuous light conditions. After 1 to 4 weeks, calli could be observed forming from the explants.

Callus derived from H. canadensis excised embryos was subcultured every 2 to 4 weeks on a defined medium, based on the result of the callus initiation experiments. Proliferating callus was selected for continued maintenance and biomass increase. In particular, callus was selected for growth in (liquid) suspension cell culture.

### Example 3

### Elite Callus Line Screening of Hydrastis canadensis for Isoquinoline Alkaloid Production

Callus was screened on a regular basis (e.g., about weekly) for production of isoquinoline alkaloids, and desirable cell lines were selected for continued proliferation. A rapid method of analysis was developed that allowed for determination of isoquinoline alkaloid (hydrastine and/or berberine) content of callus by HPLC or, a plate-based spectrophotometry assay. Further discrimination of isoquinoline alkaloids was accomplished by analyzing extracts with UHPLC separation and tandem mass detection.

A known amount of callus was selected for assay. The sample for analysis may be prepared from fresh callus or the callus may first be dried prior to extraction. Alternatively, as discussed in further detail below, a sample of the liquid medium of a suspension culture, either with or without cells, can also be used. Extraction of the fresh or dried cultured cells may involve some type of tissue disruption including freezing, sonication, the use of a bead beater, blending, centrifugation, tissue homogenization, microwaving, the use of a mortar and pestle, coffee grinder or food processor, or shearing by forcing cells through narrow bore stainless steel under high pressure. Disrupted tissue was then extracted with organic solvents to isolate the alkaloids. Solvent used may include acids, bases, alcohols, ketones, esters, fluorocarbons, hydrocarbons, organic nitriles, supercritical fluids, or any combination of these solvents.

The tissue was extracted at least one time and up to as many times as necessary to completely extract all of the alkaloids (exhaustive extraction). Further purification of the alkaloids was accomplished by solid phase extraction or other adsorption method, such as elution from a silica gel column. Extracts were combined and evaporated to dryness. Dried residue was dissolved in a minimum volume of HPLC compatible solvent, and filtered, if necessary, prior to HPLC analysis.

### Example 4

### Isoquinoline Alkaloid Analysis by HPLC with PDA and ESI-MS Detection

### HPLC Analysis

An UPLC-PDA-MS method to separate and detect the major goldenseal metabolites has been developed. This method is applicable to extracts of all goldenseal plant parts as well as to goldenseal cells in culture. The method uses a reverse-phase C-18 UPLC column and a water-acetonitrile mobile-phase gradient to separate the components of goldenseal extracts. A PDA-spectrometer and an ESI tandem mass analyzer are used to detect and characterize the components of an extract as they elute from the column.

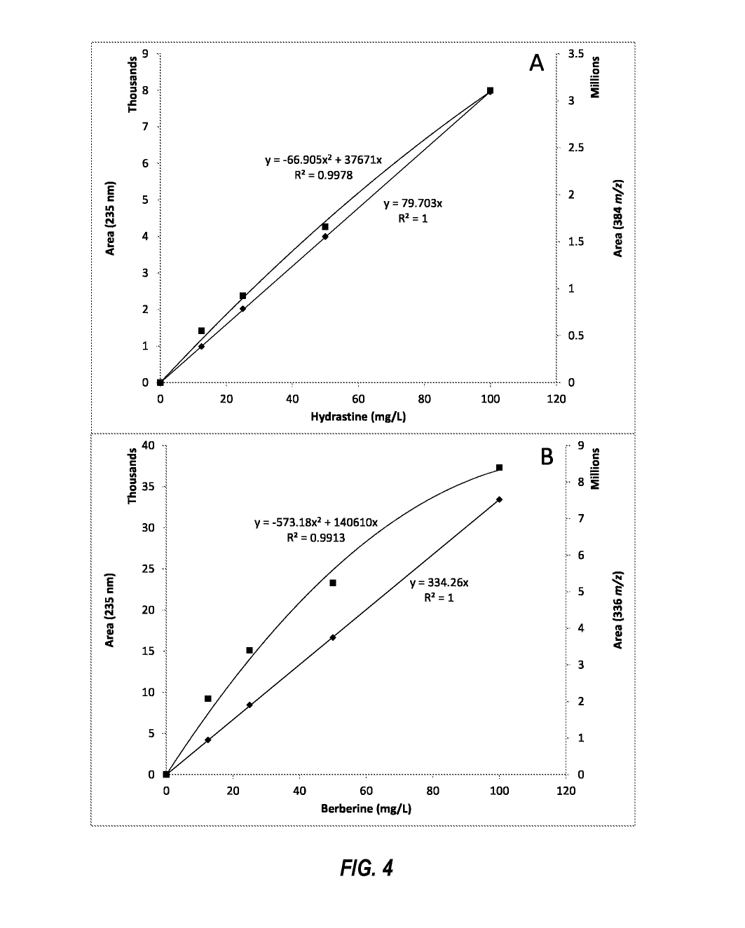

While general enough to detect a wide range of metabolites, this method focuses specific attention on detection and quantification of the high-value bioactive components of goldenseal. These include the alkaloids berberine and hydrastine. Compounds are identified by their UV and/or mass spectra and by comparison with authentic standards (e.g., commercially available, synthetic, and/or purified product. Calibration curves built from authentic standards are used for quantification (FIG. 4).

A high throughput plate based spectrophotometric method for quantification (e.g., of berberine and/or hydrastine) has also been utilized for rapid selection of cell lines. This method is based on the absorbance of an extract (e.g., at 235 nm, 440 nm, etc.) compared to a calibration curve created from authentic berberine. It has the advantage of being considerably faster and with a much higher throughput than certain LC/MS methods. As illustrated in FIG. 4, UV calibration curves for hydrastine (A) and berberine (B) are linear over typical concentration range (for extracts, whether analysis is by HPLC or plate spectrophotometer). However a second-order polynomial better fits the extracted ion mass calibration curve for each of these compounds. Without being bound to any theory, cell and/or media samples (or extracts of the (suspension) cell culture) can include compounds that absorb UV at wavelength(s) ideal and/or appropriate for measuring certain secondary metabolites, such as alkaloids, including hydrastine and/or berberine. Accordingly, because the LC/MS method detects compounds based on (charged) molecular mass, the extracted ion mass may be better suited for accurately detecting and/or quantifying compounds (e.g., secondary metabolites, such as alkaloids) that do not completely separate by HPLC.

Sample Preparation.

200 mg of fresh or 20 mg of dried freeze-dried goldenseal either from whole plant material or from cultured cells was placed in a 2 ml eppendorf tube, and 1 ml of methanol was added. The mixture was sonicated for 30 minutes then the liquid portion was passed through a 0.45 um filter. The resulting clarified extract was diluted 10-fold with methanol into a 1.5 ml sample vial for analysis.

LC/PDA/MS Analysis.

5 ul of goldenseal extract was injected using an autosampler into a Waters Acquity H-Class UPLC fitted with an Acquity UPLC BEH C18, 1.7 um, 2.1×50 mm column heated to 35 C. The mobile phase consists of a water (solvent A)/acetonitrile (solvent B) gradient each containing 0.1% formic acid. The gradient over 20 minutes is 95% A/5% B for 1 minute followed by a linear ramp to 60% A/40% B in 12 minutes then to 100% B in 20 mins followed by a 5 minute hold at 100% B. Flow rate was 0.4 ml/min. Detection was by a Waters Acquity photodiode array (PDA) detector from 210 to 750 nm followed by quadrupole mass spectrometer detection. The mass spectrometer was a Micromass Quattromicro instrument operating in electrospray positive ionization mode (ESI+) with a capillary voltage of 3 kV and a cone voltage of 20 V and with the quadrupole scanning from 100 to 600 amu. Source temperature was set to 120 C and desolvation temperature to 400 C.

### Example 5

### Isoquinoline Alkaloid Analysis by High-Throughput Spectrophotometric Analysis

Methanolic extracts of goldenseal samples are prepared in the same way as described above for LC analysis. 200 uL of each extract was added to one well of a 96-well flat-bottomed clear UV plate. Serial dilutions of a berberine calibration standard are added to a separate row of the plate. The absorbance of each well was measured at 440 nm and berberine concentrations of the samples are quantified against a calibration curve generated from the standards.

### Example 6

### Suspension Creation from Callus Derived from H. canadensis

Elite cell lines were chosen for initiation of suspensions based on their rate of growth and alkaloid production. Cell suspensions were created by introducing H. canadensis callus (prepared as in Example 2 and 3) into a liquid medium in sterile 6-well plates or Erlenmeyer flasks. The flasks were covered with silicone foam caps (or other covering that allows for oxygen exchange) and plates were sealed with micropore tape (that allowed for oxygen exchange). Plates and flasks were agitated at 50-150 revolutions per minute (rpm) in a gyrotatory shaker. The suspensions were kept in darkness or light at 18 to 30±1° C. Cell cultures were established by monitoring growth as indicated by increase in cell fresh weight or cell volume increase and by monitoring carbohydrate consumption in the medium as determined by the change in refractive index as determined by °Brix, °Plato, or specific gravity. Medium was added or changed as necessary to insure growth of the culture. Cultures were considered “established” once enough cell mass had increased to the point that a culture could be split into two subcultures of a size equal to the parent culture, and those subcultures could be subsequently subcultured (i.e. a culture was established when the “children” of the “parent” were themselves sustainable). The same variables listed in Example 2 were examined to determine the optimal growth and production media for H. canadensis cells in liquid suspension culture.

### Example 7

### Optimization of Cell Growth

This example describes methods used to increase growth of suspension cultures. Volumetric productivity of the target compounds increased as a function of the rate of cell growth and the final biomass attained when growth of the culture ended. To determine the optimal inoculum size, suspension cultures of H. canadensis cells were initiated with a cell inoculum of 5 to 50% of the culture volume, corresponding to 50 to 500 (g fresh cell weight (FCW)) L⁻¹ and grown for 3 to 21 days in different liquid medium and under different culture conditions. As detailed in Example 2, the concentrations and combinations of plant growth regulators, salts, carbohydrates, vitamins, other organic additives, agitation speed, temperature, and lighting source, intensity, and duration were tested to maximize biomass and product formation for the culture period.

In at least one embodiment, the doubling time of the suspension cell culture (or suspended cells thereof) was greater than or equal to about 1 week. For instance, the culture can increase by about 50%, 60%, 67%, 70%, 75%, 80%, 85%, 90%, 95%, 98%, 99%, 100%, in biomass in 1 week. Those skilled in the art will appreciate that doubling times less than 1 week are also contemplated herein. For instance, selection of elite cell lines can produce suspension cell cultures (or suspended cells thereof) capable of increasing by about, at least, or greater than 100%, 105%, 110%, 115%, 120%, 125%, or more in biomass in 1 week.

### Example 8

### Selection of Isoquinoline Alkaloid-Producing H. canadensis Suspension Cultures

Consumption of carbohydrates was used to monitor culture growth. Brix of suspension cultures were recorded at regular intervals from day 0 to the end of the culture cycle.

Alkaloid production (in cells and/or as released into the liquid media) was measured by HPLC, UHPLC, LC-MS analysis, and spectrophotometric analysis (see FIG. 4). Cultures or cell lines producing the greatest amount of alkaloids, or desirable ratios thereof, based on a combined metric of cell growth and alkaloid production, were selected for subsequent transfer (or subculturing). Cultures or cell lines exhibiting undesirable growth and/or production indicators (e.g., decreased alkaloid production) were discarded in favor of better growing and/or higher producing cultures or cell lines. Discarded cultures or cell lines often exceeded the number of transferred cultures or cell lines. However, long-term and/or strong selective pressure required perseverance through less than ideal culture periods and/or cell lines. Some transfers included passaging of cultures that did not exhibit a significant increase in growth and/or production, but which did not exhibit decreased alkaloid production. Other transfers included passaging of cultures or cell lines exhibiting a decrease in one alkaloid and an increase in another alkaloid. Generally, cell selection included frequently (e.g., weekly) and repeatedly monitoring alkaloid production in multiple suspension cell subcultures and passaging (or subculturing) suspension cell cultures producing desirable amounts and/or ratios of alkaloids.

Elite cultures were further selected by analysis of the top 10% to 15% of cultures selected for subculture by extended HPLC and HPLC-MS methods to select for those cultures that produce the greatest concentration of alkaloids, or desirable ratios thereof. Analyzed isoquinoline alkaloids were harvested, isolated, purified, and/or extracted from the cells of H. canadensis culture and/or from medium in which the suspension cells of H. canadensis culture were grown.

Other cultures were selected to produce suspension cells having an increased concentration or amount of one or more (isoquinoline) alkaloids. For instance, suspension cells or cell lines were subjected to multiple rounds of selection to optimize the level of isoquinoline alkaloid(s) produced by or in the cells and, optionally, released into the liquid culture medium. Specific methods included selecting cells that (1) retain optimal levels of isoquinoline alkaloid within the cells of the suspension culture and/or (2) release optimal levels of isoquinoline alkaloid into the medium of the suspension culture.

Some cultures were selected to produce suspension cells having an altered and/or desirable ratio of (isoquinoline) alkaloids. For instance, suspension cells or cell lines were subjected to multiple rounds of selection to optimize the ratio of isoquinoline alkaloids (e.g., hydrastine:berberine) produced by or in the cells. Specific methods included selecting cells that (1) retain an optimal ratio of isoquinoline alkaloids within the cells of the suspension culture and/or (2) release an optimal ratio of isoquinoline alkaloid into the medium of the suspension culture.

### Example 9

### Optimization of Isoquinoline Alkaloid Production by H. candensis Suspensions Using a Two-Stage Culture Method

The feasibility of optimizing culture growth and isoquinoline alkaloid production using a two-stage process was investigated. In this method, the first stage involved growing cells to a maximum density by optimizing conditions for cell growth, then replacing all or part of the spent culture medium with a second medium that has been optimized for production of the target compound. Isoquinoline alkaloid production in suspension cells and/or content in culture media were monitored through successive rounds of selection.

### Example 10

### Elicitation of Isoquinoline Alkaloid Production in H. canadensis Cell Cultures

Both biotic and abiotic compounds were tested for their influence on the production of isoquinoline alkaloids in culture (e.g., cellular and/or liquid media content). Concentration, combination, and timing of elicitor addition to the culture were tested. Effectiveness of elicitation was evaluated by measuring growth of the culture and production of each of the alkaloids in suspension cells and/or culture media. Statistical software was used for the design and analysis of the experiments.

### Example 11

### Extraction of Isoquinoline Alkaloids from H. canadensis Suspension Cultures

This example describes methods developed for extracting isoquinoline alkaloids from suspension cells of H. canadensis cultures developed in examples 1 to 8.

For LC-MS analysis, a portion of the residue was suspended in methanol, centrifuged and filtered through a 0.1 to 0.45 um membrane filter. If necessary, the extract was diluted ten-fold with acetonitrile. Concentrated extracts were stored at −20° C. in methanol and under nitrogen to prevent degradation (e.g., oxidative degradation).

In some embodiments, isoquinoline alkaloids can be harvested, isolated, purified, and/or extracted from medium in which the suspension cells of H. canadensis culture are grown.

### Example 12

### Scale-Up of H. canadensis Suspension Cultures

A common problem in the use of plant cell cultures is the consistent and stable production of target products. Therefore, a key for successful large-scale plant cell culture was to maintain stable productivity. A process to scale-up suspensions of cell cultures from 6-well plates to 4000 mL flasks was successfully developed. The speed of the shakers was optimized for all flasks to give similar growth and productivity regardless of the size of the culture or culture vessel. Agitation rates of 40 to 150 RPM were examined and DO (dissolved oxygen) levels were monitored and compared. At regular intervals during the culture cycle, biomass, sugar concentration in medium, and alkaloid content was measured. An optimal agitation rate and culture volume was determined that provided satisfactory culture growth, productivity, and DO in all flask sizes. Various parameters, including agitation rates between 0 and 400 RPM, temperature between 4 and 105 degrees C., and light exposure between 0 and 24 hours per day can be altered to further optimize the production of isoquinoline alkaloids (or ratio thereof) in certain embodiments. In addition, large-scale bioreactors (e.g., of up to or greater than 55 liters) can also be utilized in certain embodiments.

### Example 13

### Amounts and Ratios of Isoquinoline Alkaloids in H. canadensis Suspension Cells and Commercial Rhizomes

This example describes comparative analytical results of isoquinoline alkaloids extracted from suspension cells of H. canadensis cultures and from commercial rhizomes.

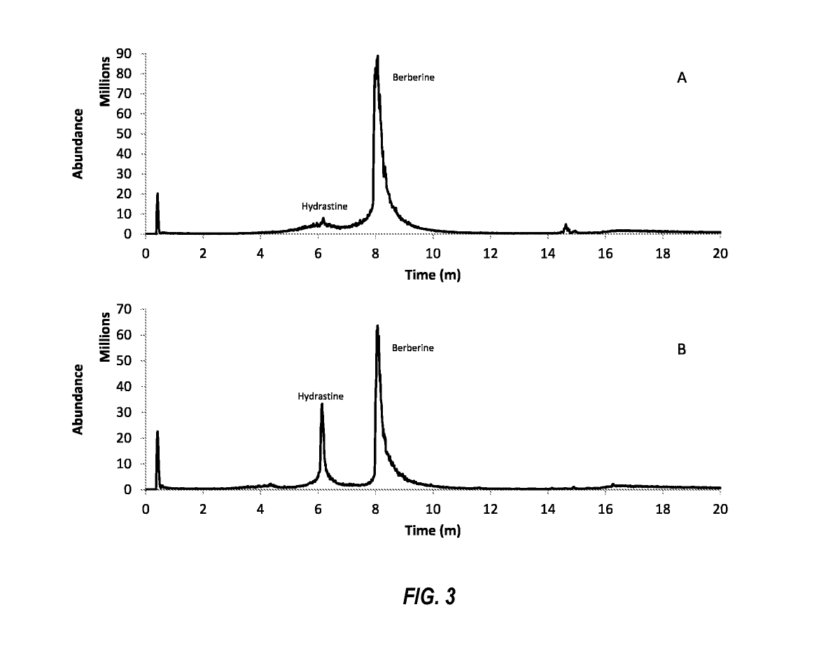

Extract from suspension (callus) cells derived from H. canadensis and sampled shortly after initiation of cultures and before selection had begun (FIG. 3A) and commercial rhizomes of H. canadensis (FIG. 3B) were analyzed by UV detection (at 235 nm) and/or extracted ion (HPLC-MS) detection for determination of berberine (336 m/z) and hydrastine (384 m/z) content, and hydrastine:berberine ratio. The area under each peak can be determined by known methods and the value matched against the respective calibration curve(s), as illustrated in FIG. 4, to accurately determine the concentration of alkaloid(s) in the sample. However, it should be apparent to those skilled in the art (from FIGS. 3A and 3B) that unselected callus suspension cells do not produce hydrastine at the ratio with berberine that is found in the natural plant or rhizomes thereof. Cellular hydrastine levels (amounts) could not be accurately quantified in unselected callus. Accordingly, to achieve USP approved amounts of hydrastine and/or berberine, or a suitable ratio of hydrastine to berberine in a goldenseal natural product (e.g., freeze-dried suspension cells or extract thereof), hydrastine production needed to be increased (e.g., through generational selection of cultures with improved hydrastine production).

Without being bound to any theory, generally, peak height can be used for a rough comparison of alkaloid content between samples and/or an estimation of alkaloid content in a sample (assuming the same type and amount of starting material and the same dilution factor between compared samples, or calculations to correct for differences). However, comparison between samples provided at different starting amounts, samples prepared using different dilution factors, and/or samples of different material (e.g., rhizome content vs. callus cell content vs. liquid media content) is much less accurate. Exact measurements may require computer-aided determination of the (corrected) peak area. Moreover, rhizomes or other goldenseal plant tissues are not the same as undifferentiated callus cells. It will be appreciated that alkaloid production and selection in callus derived from goldenseal is highly unpredictable.

### Example 14

### Analysis of H. canadensis Suspension Culture Media

This example describes methods developed for analyzing isoquinoline alkaloid content in H. canadensis suspension cell culture media.

Suspension cell cultures of H. canadensis were unexpectedly selected to release cellular metabolites (e.g., isoquinoline alkaloids) into the culture medium. Rather than using methanol extraction (or another extraction technique known in the art) from the cells, analysis can be conducted following addition of an appropriate amount of acetonitrile, methanol, or other solvent to a sample of the liquid culture media. UV (absorption) and/or MS can be used to determine concentration and/or ratio of isoquinoline alkaloids in (or released by the suspension cells into) the liquid culture media.

Alkaloids and other compounds of interest that have been excreted into the culture medium may be harvested by several techniques. For instance, solid phase extraction (SPE) can be an effective means of isolating organic compounds dissolved in the culture medium by adsorbing them onto a solid resin. This can be accomplished either by the direct addition of a resin to the culture medium followed by isolation of the resin by filtration, or by passing the medium through a plug or column of resin. In either case, the adsorbed compounds can subsequently be eluted from the solid phase using methanol, ethanol, or other suitable solvent. The solid phase may be an ion-exchange resin such as Amberlite XAD or other polymeric adsorbent, or any other functionalized silica resin such as C-18.

Alternatively, compounds of interest can be isolated from media by liquid-liquid (e.g., aqueous two-phase) extraction, for example using dichloromethane, ethyl acetate, ether, or any other (water-immiscible and/or organic) solvent. Compounds can then be recovered upon evaporation of the solvent. Freeze-drying can also be used to remove water from the medium and isolate compounds that were present in solution.

In addition, it will be appreciated that neither goldenseal plants nor callus derived therefrom were known to release cellular (secondary) metabolites (e.g., isoquinoline alkaloids). Accordingly, the selection of callus cells that release isoquinoline alkaloids, alone, was not only unpredictable, but advantageous. For instance, callus cells that release isoquinoline alkaloids can be selected to grow in suspension cell culture. grown for extended periods of time in suspension cell culture, over which time the isoquinoline alkaloids are released into the medium.

### Example 15

### Amounts and Ratios of Isoquinoline Alkaloids in H. canadensis Suspension Culture Media

This example describes analytical results of isoquinoline alkaloid content and/or ratio produced by suspension cells of H. canadensis and released into the culture media thereof.

Suspension (callus) cells derived from H. canadensis can be selected to produce one or more isoquinoline alkaloid(s) (e.g., hydrastine and berberine) in amounts and/or at ratios that are different than (i.e., greater than or less than) those produced by or found in goldenseal (commercial) rhizomes, native plants, plant parts, plant cells, and/or unselected (or less selected) callus cells, from which the selected cells were derived. For instance, suspension cultures of goldenseal-derived callus cells can be selected to produce a higher ratio of berberine to hydrastine than produced by or found in (commercial) rhizomes and/or native plant cells.

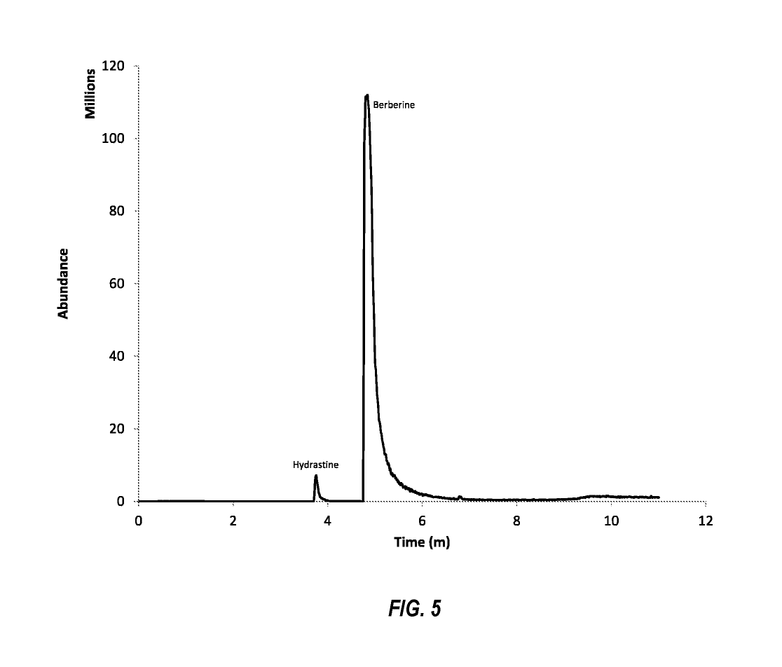

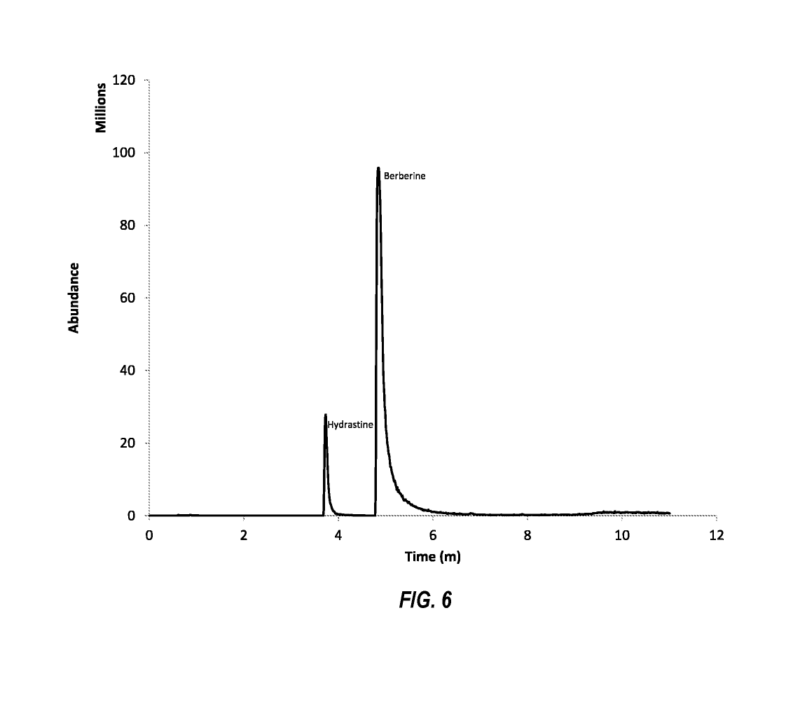

High berberine cell lines were selected and cultured to produce and release the isoquinoline alkaloids hydrastine and berberine at a hydrastine:berberine ratio of about 1:3 (FIG. 5) and 1:60 (FIG. 6) into the liquid culture medium (on a molar, w/w, and/or w/v basis). Surprisingly, the 1:60 cell line (FIG. 6) also produced and released berberine into the liquid culture medium at a concentration of about 2.3 grams per liter of culture and/or liquid culture medium (e.g., in a 1 week culture). Accordingly, goldenseal (callus) cell lines can be selected to produce (and release into the liquid culture medium) up to at least 2.3 grams of berberine per liter of liquid medium per week in suspension cell culture. Thus, high berberine cell lines can be selected to produce (and release) a high concentration or amount of berberine and a high ratio with hydrastine (compared to ratios found in native plants or plant parts, (commercial) rhizomes, or extracts thereof and/or unselected (or less selected) callus cells, from which the selected cells were derived).

High berberine cell lines can also be selected to produce and release a high concentration or amount of hydrastine into the liquid culture medium. For instance, at least one cell line was selected to produce and release about 282 mg berberine and about 64 mg hydrastine per liter of liquid culture medium per week (or in a one week culture). Accordingly, the ratio of hydrastine to berberine released into the medium by high berebrine producing cell lines can be about 1:4.4 (or 64:282) in some embodiments.

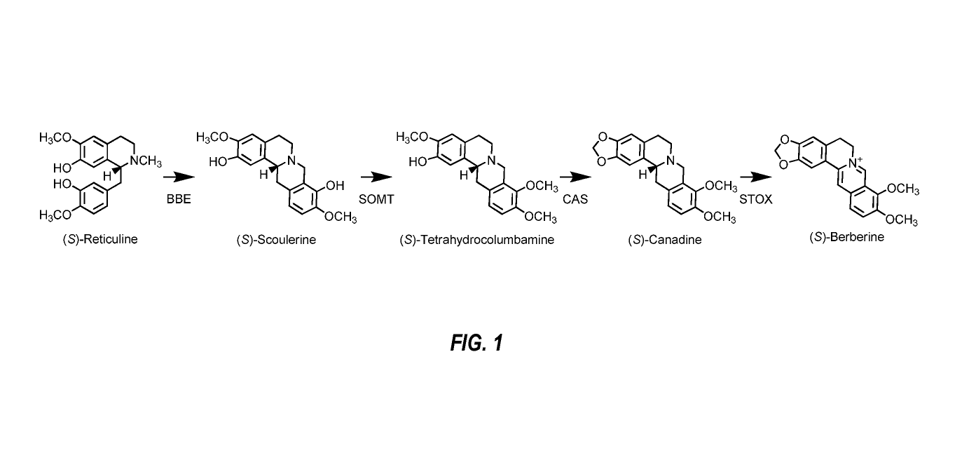

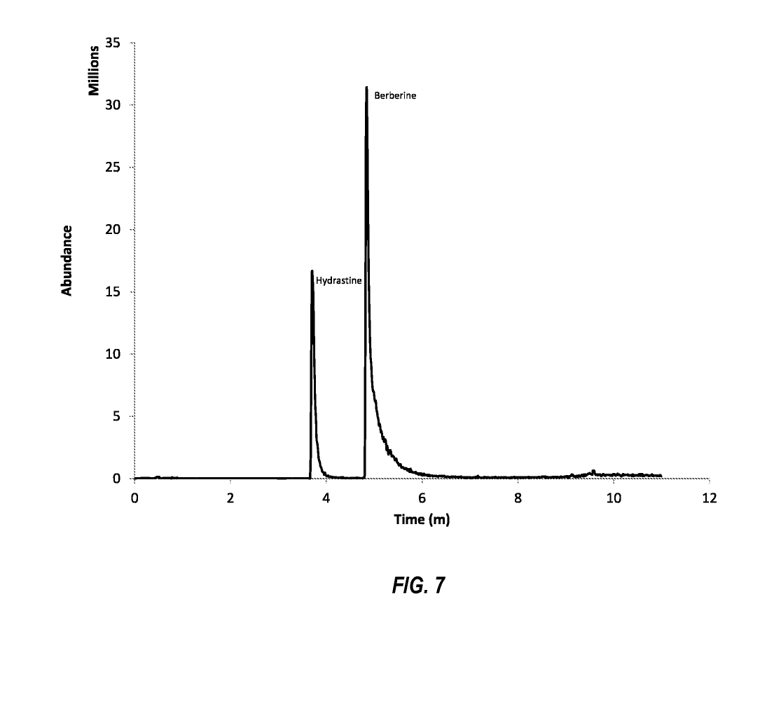

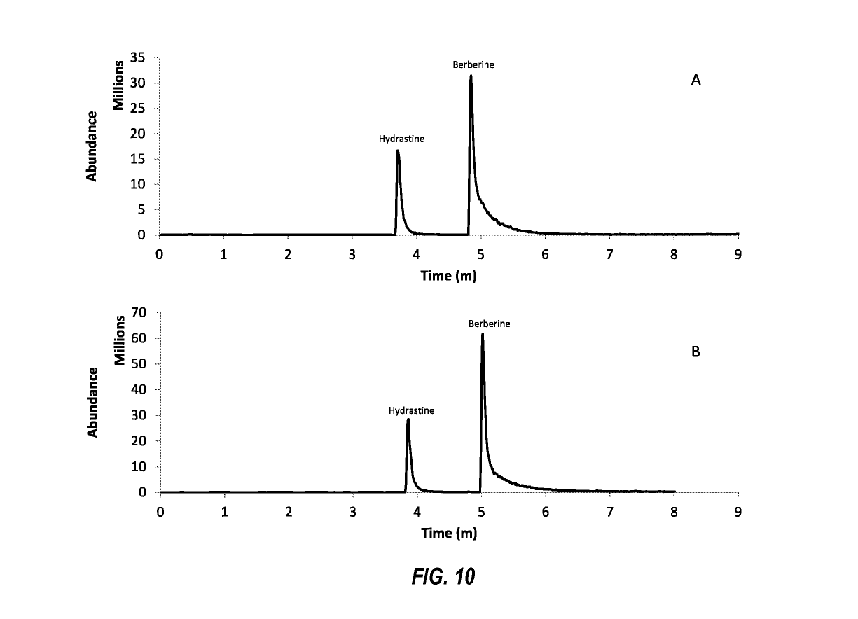

Suspension cultures of goldenseal-derived callus cells can also be selected to produce a higher concentration of hydrastine, or ratio of hydrastine to berberine, than produced by or found in native plants or plant parts, (commercial) rhizomes, or extracts thereof and/or unselected (or less selected) callus cells, from which the selected cells were derived. For instance, at least one cell line was selected to produce and release hydrastine and berberine at a hydrastine:berberine ratio of about 1:1 (on a molar, w/w, and/or w/v basis) into the liquid culture medium (FIG. 7). Without being bound to any theory, it is noted that the 1:1 ratio of hydrastine to berberine illustrated in FIG. 7 shows a substantially higher berberine chromatogram peak than the hydrastine peak. As depicted in FIG. 1, however, berberine is protonated, having a positively-charged amine group in its isoquinoline group. Accordingly, it is believed that berberine ionizes more completely under these conditions than hydrastine, resulting in the larger berberine peak. Thus, cell lines can be selected to produce (and release) an approximately equal ratio of berberine and hydrastine (into the liquid culture medium). As illustrated in FIG. 10, goldenseal cells from suspension (A), selected for a hydrastine:berberine ratio of 1:1 based on quantified mass, have a similar ratio of hydrastine:berberine as commercial goldenseal rhizomes (B).

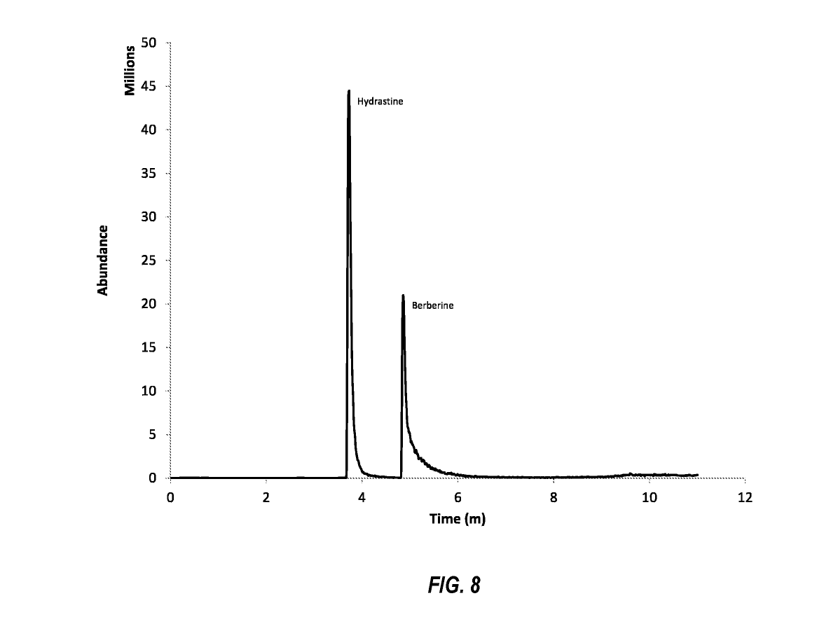

A high hydrastine cell line was also selected to produce and release hydrastine and berberine at a hydrastine:berberine ratio of about 6:1 (on a molar, w/w, and/or w/v basis) into the liquid culture medium (FIG. 8). Surprisingly, this cell line also produced and released hydrastine into the liquid culture medium at a concentration of about 180 milligrams per liter of culture and/or liquid culture medium (e.g., in a 1 week culture). However, goldenseal (callus) cell lines can and/or have been be selected to produce (and release into the liquid culture medium) up to at least 326 milligrams of hydrastine per liter of liquid medium per week in suspension cell culture. High hydrastine cell lines can also be selected to produce and release a high concentration or amount of berberine (e.g., up to at least about 234 mg/L berberine) into the liquid culture medium. Accordingly, the ratio of hydrastine to berberine released into the medium by high hydrastine producing cell lines can be about 1.4:1 (or 326:234) in some embodiments.

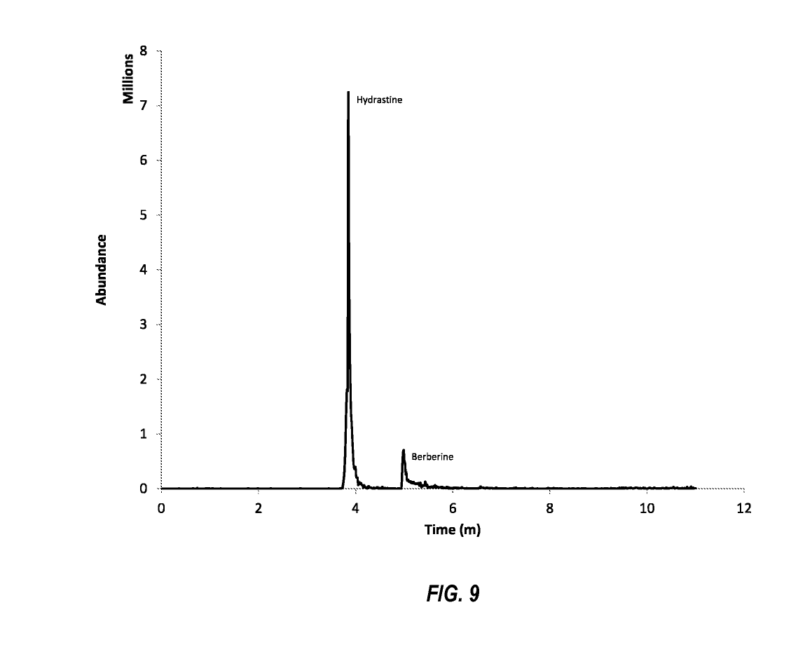

An even higher hydrastine cell line was selected to produce and release hydrastine and berberine at a hydrastine:berberine ratio of about 16:1 (on a molar, w/w, and/or w/v basis) into the liquid culture medium (FIG. 9). Thus, the ratio of hydrastine to berberine can be further altered (e.g., increased or decreased) through cell selection to achieve ratios of hydrastine to berberine (in the cells, liquid culture medium, or extract) different than ratios found in native plants or plant parts, (commercial) rhizomes, or extracts thereof and/or unselected (or less selected) callus cells, from which the selected cells were derived.

It is noted that embodiments of the present disclosure can include non-genetically modified (non-GMO) cells. In particular, certain embodiments include cells that are or have been selected for altered production of one or more secondary metabolites without introducing and/or applying any heterologous or foreign genetic material(s) and/or known mutagen(s) to or into the cells, (suspension) cell culture, medium, etc. Accordingly, products of the present disclosure, including (selected and/or elite) cells or cell lines, suspension cells and/or cell cultures, natural products and/or natural product preparations, extracts, etc. can be or comprise non-GMO products in one or more embodiments.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent, or having results or functions similar or equivalent to those described herein can be used to practice embodiments of the present disclosure, only certain components and methods are described herein.

It will also be appreciated that systems, processes, and/or products according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure. Moreover, unless a feature is described as requiring another feature in combination therewith, any feature herein may be combined with any other feature of a same or different embodiment disclosed herein. Furthermore, various well-known aspects of illustrative systems, processes, products, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

We claim:

1. A method of producing an isoquinoline alkaloid natural product preparation, comprising:
establishing callus derived from tissue of a natural plant that produces one or more isoquinoline alkaloids;
selecting a cell line of callus cells with increased production of the one or more isoquinoline alkaloids compared to the tissue of the natural plant from which the cell line was derived;
cultivating the selected callus cells in a liquid medium to obtain a suspension cell culture; and
recovering natural product from the selected callus cells, liquid medium, or both, of the suspension cell culture, thereby producing a natural product preparation with increased isoquinoline production.

2. The method of claim 1, wherein the natural plant is goldenseal (Hydrastis canadensis), the callus comprising goldenseal plant callus.

3. The method of claim 1, wherein the one or more isoquinoline alkaloids are selected from the group consisting of berberine and hydrastine.

4. The method of claim 1, wherein the selected callus cells produce hydrastine and berberine at a ratio other than that found in the tissue of the natural plant from which the callus was derived.

5. The method of claim 1, wherein the selected callus cells produce hydrastine and berberine at a ratio of greater than or equal about 2:2.5, preferably 2:2.25, more preferably 1:1, still more preferably 1.5:1, still more preferably 1.6:1, still more preferably 2:1, still more preferably 2.5:1, still more preferably 3:1, still more preferably 3.5:1, still more preferably 4:1, still more preferably 4.5:1, still more preferably 5:1, still more preferably 5.5:1, still more preferably 6:1, hydrastine:berberine.

6. The method of claim 1, wherein the selected callus produce hydrastine and berberine at a ratio of less than or equal to about 2:2.5, preferably 1:1.5, more preferably 1:2, still more preferably 1:5, still more preferably 1:10, still more preferably 1:25, still more preferably 1:50, still more preferably 1:60, hydrastine:berberine.

7. The method of claim 1, further comprising selecting the callus cells to release the one or more isoquinoline alkaloids into the liquid medium in suspension cell culture, the selected cell line releasing the one or more isoquinoline alkaloids into the liquid medium.

8. The method of claim 7, wherein recovering natural product comprises recovering natural product from the liquid medium, the liquid medium having:
at least about 1 mg/L, 2 mg/L, 3 mg/L, 5 mg/L, 8 mg/L, 10 mg/L, 15 mg/L, 20 mg/L, 25 mg/L, 30 mg/L, 35 mg/L, 40 mg/L, 45 mg/L, 50 mg/L, 55 mg/L, 60 mg/L, 64 mg/L, 80 mg/L, 100 mg/L, 150 mg/L, 200 mg/L, 250 mg/L, 275 mg/L, 300 mg/L, 325 mg/L, or 326 mg/L hydrastine;
at least about 1 mg/L, 2 mg/L, 3 mg/L, 5 mg/L, 8 mg/L, 10 mg/L, 15 mg/L, 20 mg/L, 25 mg/L, 30 mg/L, 35 mg/L, 40 mg/L, 45 mg/L, 50 mg/L, 55 mg/L, 60 mg/L, 100 mg/L, 150 mg/L, 200 mg/L, 234 mg/L, 250 mg/L, 282 mg/L, 300 mg/L, 500 mg/L, 1 g/L, 2 g/L, or 2.3 g/L berberine; or
both.

9. The method of claim 7, further comprising selecting the callus cells to produce and release into the liquid medium:
hydrastine at greater than or equal to about 1 mg/L, 2 mg/L, 3 mg/L, 5 mg/L, 8 mg/L, 10 mg/L, 15 mg/L, 20 mg/L, 25 mg/L, 30 mg/L, 35 mg/L, 40 mg/L, 45 mg/L, 50 mg/L, 55 mg/L, 60 mg/L, 64 mg/L, 80 mg/L, 100 mg/L, 150 mg/L, 200 mg/L, 250 mg/L, 275 mg/L, 300 mg/L, 325 mg/L, or 326 mg/L of liquid medium;
berberine at greater than or equal to about 1 mg/L, 2 mg/L, 3 mg/L, 5 mg/L, 8 mg/L, 10 mg/L, 15 mg/L, 20 mg/L, 25 mg/L, 30 mg/L, 35 mg/L, 40 mg/L, 45 mg/L, 50 mg/L, 55 mg/L, 60 mg/L, 100 mg/L, 150 mg/L, 200 mg/L, 234 mg/L, 250 mg/L, 282 mg/L, 300 mg/L, 500 mg/L, 1 g/L, 2 g/L, or 2.3 g/L of liquid medium; or
both.

10. The method of claim 1, wherein the callus cells are selected to produce and retain:
at least about 1%, 1.5%, 2%, 2.5%, 3%, 3.5%, 4%, 4.5%, 5%, 5.5%, or 5.6% hydrastine on a dry weight basis;
at least about 1%, 1.5%, 2%, 2.5%, 3%, or 3.5% berberine on a dry weight basis; or
at least about 4.5%, 5%, 5.5%, 5.6%, 6%, 6.5%, 7%, 7.5%, 8%, 8.5%, 9%, or 9.1% total hydrastine plus berberine on a dry weight basis.

11. The method of claim 1, wherein the liquid medium comprises one or more components selected from the group consisting of a carbohydrate source, a nitrogen source, major salts, minor salts, and one or more hormones selected from the group consisting of an auxin and a cytokinin.

12. A method of producing an isoquinoline alkaloid natural product preparation, comprising:
establishing callus derived from tissue of goldenseal (Hydrastis canadensis);
selecting a cell line of callus cells to grow in suspension cell culture, produce one or more isoquinoline alkaloids produced by goldenseal plants, and release the one or more isoquinoline alkaloids;
cultivating the selected callus in a liquid medium to obtain a suspension cell culture, the selected callus growing in the suspension cell culture, producing one or more isoquinoline alkaloids produced by goldenseal plants, and releasing the one or more isoquinoline alkaloids into the liquid medium; and
recovering natural product from the liquid medium, thereby producing a natural product preparation with isoquinoline alkaloid.

13. The method of claim 12, wherein the one or more isoquinoline alkaloids include berberine and/or hydrastine.

14. The method of claim 12, wherein the callus cells are selected to release the one or more isoquinoline alkaloids into the liquid medium at a ratio other than the ratio at which the one or more isoquinoline alkaloids are found in the tissue from which the callus was derived.

15. The method of claim 12, wherein the callus cells are selected to produce and release, or produce and retain, an increased amount of the one or more isoquinoline alkaloids compared to the amount found in the tissue from which the cell line was derived.

16. An isolated cell line, comprising:
callus cells derived from tissue of goldenseal (Hydrastis canadensis), the callus cells being selected to:
grow in suspension cell culture;
produce berberine and hydrastine; and
release the berberine and hydrastine when the callus cells are grown in liquid medium comprising a carbohydrate source, a nitrogen source, major salts, minor salts, and one or more hormones selected from the group consisting of an auxin and a cytokinin.

17. The cell line of claim 16, wherein the callus cells are selected to produce hydrastine and berberine at a ratio other than that found in the tissue of the natural plant from which the callus was derived.

18. The cell line of claim 16, wherein the callus cells are selected to produce an increased amount of berberine, hydrastine, or both, as compared to the tissue from which the callus cells were derived.

19. A suspension cell culture, comprising:
the cell line of claim 16; and
a liquid medium comprising a carbohydrate source, a nitrogen source, major salts, minor salts, and one or more hormones selected from the group consisting of an auxin and a cytokinin,
the cell line growing in the liquid medium, producing one or more isoquinoline alkaloids, and releasing the one or more isoquinoline alkaloids into the liquid medium.

20. An extract of the suspension cell culture of claim 19, comprising greater than 2% hydrastine on a dry weigh basis.
